(19) United States
(12) Patent Application Publication
(10) Pub. No.: US 2020/0032242 A1
(43) Pub. Date: Jan. 30, 2020

(54) MULTI-STEP PROCESSING DEVICE FOR NUCLEIC ACID EXTRACTION

(71) Applicant: Paratus Diagnostics, LLC, Austin, TX (US)

(72) Inventor: Roland Schneider, Austin, TX (US)

(21) Appl. No.: 16/526,771

(22) Filed: Jul. 30, 2019

Related U.S. Application Data
(60) Provisional application No. 62/712,120, filed on Jul. 30, 2018.

(51) Int. Cl. C12N 15/10

(52) U.S. Cl. CPC C12N 15/1006; C12N 15/1017

(57) ABSTRACT

The present disclosure relates to sample processing device and associated methods of use. The sample processing device includes an internal chassis comprising one or more receiving chamber and one or more corresponding rows of buffer chambers operable to store a buffer liquid. The device also includes an external chassis having an actuator that is operable to actuate a piston. The piston is in turn operable to sequentially engage each buffer chamber of each row of buffer chambers as the internal chassis moves through a series of index positions relative to the external chassis. When actuated, the piston causes the dispensation of fluid from the underlying buffer chambers to the corresponding receiving chambers. A valve is coupled to the internal chassis and operable to sequentially provide a fluid coupling between the respective receiving chambers and each buffer chamber of the corresponding rows of buffer chambers. The device also includes a lever that is operable to move the internal chassis relative to the external chassis though a plurality of index positions in response to the actuator being activated.

### TECHNICAL FIELD

The present disclosure relates generally to the field of medical diagnostics and more particularly to in vitro medical diagnostic devices including point-of-care in vitro medical diagnostic devices.

### BACKGROUND OF THE INVENTION

There is a recognized and compelling need for the rapid and accurate analysis of biological samples in an outpatient setting. Multitudes of devices have already been brought to market to analyze biological samples for the purposes of treating patients and infection diseases. However, most such devices require a laboratory environment and resources, including access to wired computer networks, stationary laboratory equipment, and on-site electrical power. The testing for infectious pathogens in human patient specimens is therefore largely confined to centralized laboratory testing in Clinical Laboratory Improvement Amendment (CLIA) rated medium-complexity or high-complexity facilities. Commonplace techniques used in such laboratories include traditional culturing of specimens, immunological assaying using Enzyme-Linked Immunosuppressant Assay (ELISA), nucleic acid testing (such as polymerase chain reaction, PCR), and other methods.

### SUMMARY

In an illustrative embodiment, a method of preparing a nucleic acid specimen includes depositing a lysed DNA sample to a DNA receiving chamber of a nucleic acid processing device, the nucleic acid processing device includes an actuator and a first row of buffer chambers. The method further includes activating the actuator of the of the nucleic acid processing device at a first time. Actuation of the actuator at the first time causes a first liquid to be propelled from a first chamber of the first row of buffer chambers to the DNA receiving chamber. The method further includes activating the actuator of the nucleic acid processing device at a second time, wherein actuation of the actuator at the second time causes a second liquid to be propelled from a second buffer chamber of the first row of buffer chambers to the DNA receiving chamber.

In another illustrative embodiment, a sample processing device includes an internal chassis having a first receiving chamber and a first row of buffer chambers. The sample processing device also includes an external chassis and an actuator. The actuator is operable to actuate a piston that is operable to engage each buffer chamber of the first row of buffer chambers. The sample processing device further includes a valve coupled to the internal chassis. The valve is operable to fluidly couple the first receiving chamber to a first buffer chamber of the first row of buffer chambers when the valve is in a first orientation, and to a second buffer chamber when the valve is in a second orientation. The sample processing device has a lever that is operable to move the internal chassis relative to the external chassis though a plurality of index positions in response to the actuator being activated. In addition, the actuator is operable to cause a first liquid to be propelled from the first buffer chamber to the first receiving chamber when actuated at a first time.

### BRIEF DESCRIPTION OF THE DRAWINGS

FIG. 1 is a schematic, perspective view of an illustrative embodiment of a specimen delivery cartridge;

FIG. 2a is a schematic, perspective view of an alternative embodiment of a specimen delivery cartridge;

FIG. 2b is a composite view showing a stool-swab before and after a specimen was collected form the swab by roiling;

FIG. 3 is a perspective view of an internal chassis of a multi-step processing device;

FIG. 4 is a perspective view of the multi-step processing device, wherein the internal chassis is inserted into an external chassis of the multi-step processing device for processing;

FIG. 5 is a section view of the multi-step processing device, taken along section lines 5-5 shown in FIG. 4;

FIG. 6 is a bottom view of the internal chassis of FIG. 3; and

FIGS. 7-9 are perspective views of a pivotable valve in accordance with an illustrative embodiment.

### DETAILED DESCRIPTION

The present disclosure relates to a biological sample processing system that is designed to meet the need for a low-cost nucleic acid extraction device. In an illustrative embodiment, the system does not require electric power or external instrumentation and is suitable for use in resource-poor settings. The device may alternatively be used to process any type of specimen, or even multiple specimens in parallel, in each case in a manner that enables sequential, multi-step processing. As such, it is noted that while the following disclosure references to the processing of nucleic acid samples, such references should be understood in most instances to be similarly applicable to processing other types of samples and specimen.

In an illustrative embodiment, a specimen processing cartridge is paired with a dual channel, push-button device that includes mechanisms for solid-phase extraction of one or more specimens. The specimens may be DNA (deoxyribonucleic acid) and RNA (ribonucleic acid). Both parts of the system may function without electric power and could be combined into a single compact unit with auto-advancing functionality that further minimizes user involvement.

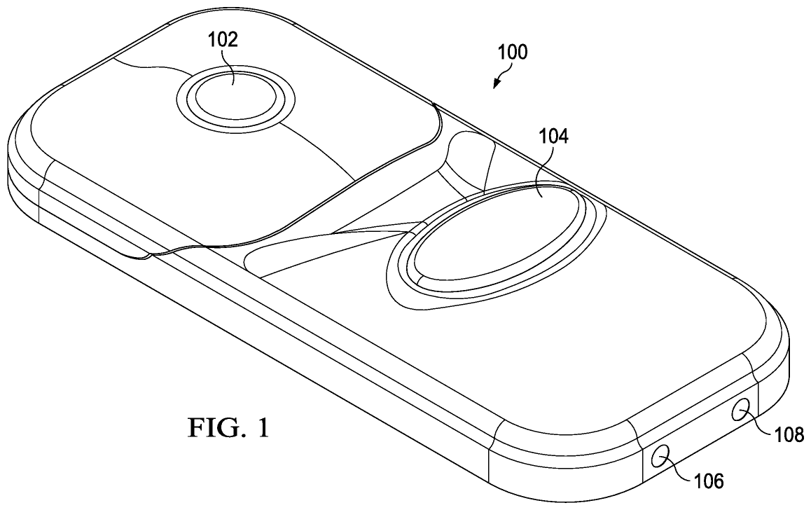

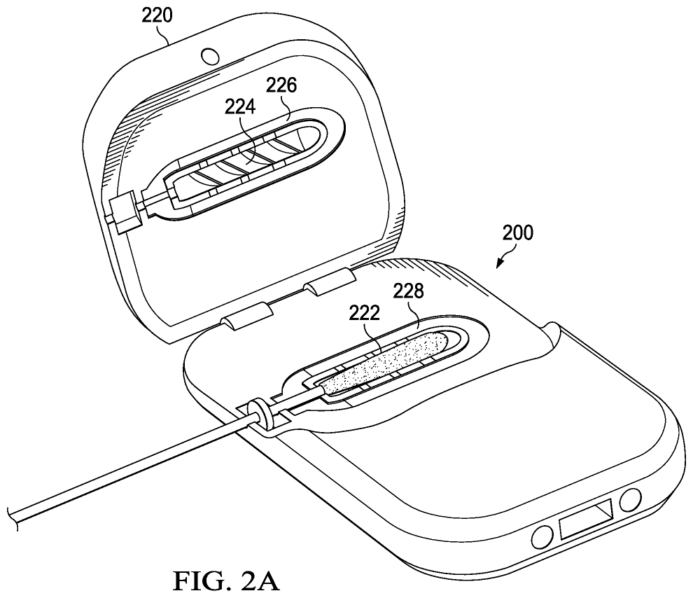

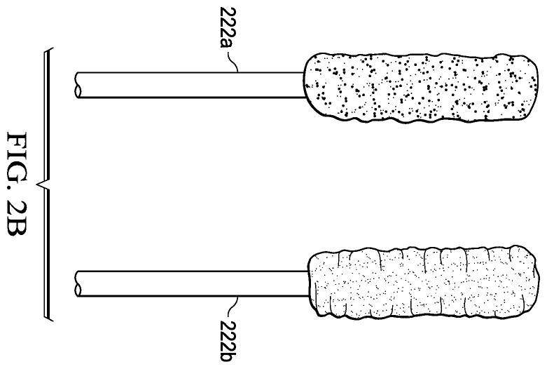

FIG. 1 is an illustration of a specimen processing cartridge 100 in accordance with an illustrative embodiment. The specimen processing cartridge 100 houses an internal roiling structure and solid-phase extraction system. The specimen processing cartridge 100 may be operated to extract a sample from a swab or an alternative sample collection device. The specimen processing cartridge 100 includes a manual actuator 102, in the form of a button, that may be depressed to force an elution liquid across the roiling structure (similar to roiling mechanism 224 of FIG. 2) to extract a specimen (e.g., a biological specimen) from the sample collection device. The specimen processing cartridge 100 also includes a second actuator 104, which may be pressed multiple times to release a nucleic acid extraction/wash liquid to interact with at least a portion of the elution liquid that includes the extracted specimen in solution or suspension, as described in more detail below. The sample processing device may include internal buffer chambers (e.g., a DNA sample buffer chamber and a RNA sample buffer chamber), which may in turn be coupled to outlet ports (DNA outlet port 106 and RNA outlet port 108).

Referring now to FIG. 2A, a representative specimen processing cartridge 200 contains a unique, roiling mechanism, which creates a vortexing fluid stream around a sample collection swab 222 to rapidly and efficiently lyse and recover a biological sample from a variety of specimen types. Examples of specimen that may be processed include saliva, stool, whole blood, and nasopharyngeal swabs. No electric power, pumps, or external instruments may be needed for the specimen processing cartridge 200 to function. The user simply inserts the swab, closes the lid, and presses the elution button.

The exemplary specimen processing cartridge 200 includes a lid 220, which is shown in the open position to reveal the location of the swab 222 and internal structure of the roiling mechanism 224. A lysis buffer solution is pre-loaded within a reservoir in the lid and dispensed into the roiling mechanism 224 when the elution button is pushed, thereby enabling simultaneous chemical and mechanical lysis of the sample collected from the swab 222 via shear stress as the elution circulates about the sample collection swab (or a similar sample collection device).

Silicone gaskets 226, 228 with interlocking structures provide a reliable, leak-proof seal once a user closes the specimen processing cartridge 200. The design of the specimen processing cartridge 200 thereby also ensures that the lid 220 is easily closed without the use of excessive force. Effectiveness of the roiling mechanism 224 is illustrated in FIG. 2B, which shows on the left-hand side an undiluted stool specimen that was collected onto a flocked swab 222a (Copan FLOQSwab™) and on the right-hand side, the flocked swab 222b after processing by the specimen processing cartridge 200. Before roiling, the swab was dark brown and after roiling the swab is nearly clean, indicating that the sample has been successfully lysed, recovered from the swab, and made ready for downstream purification.

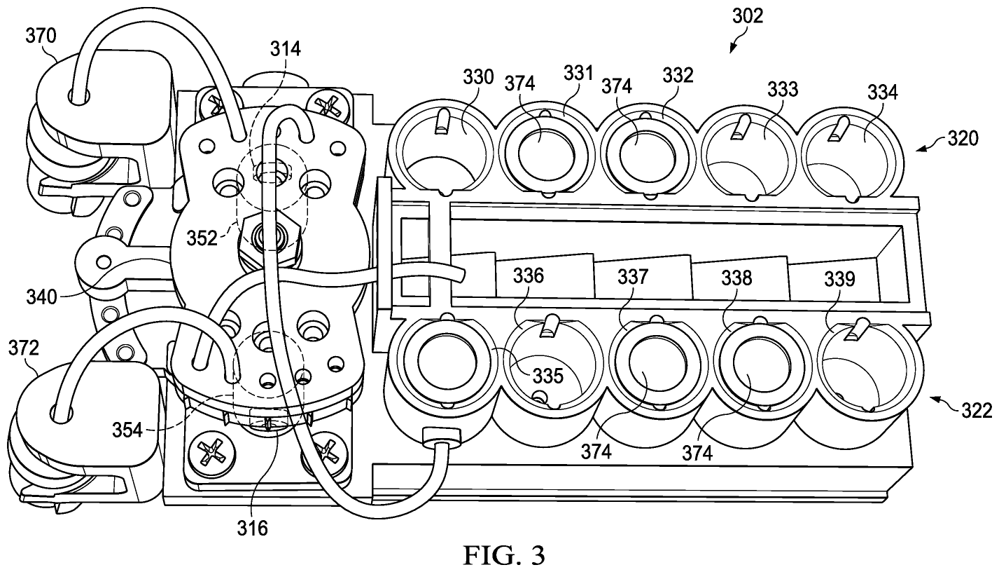

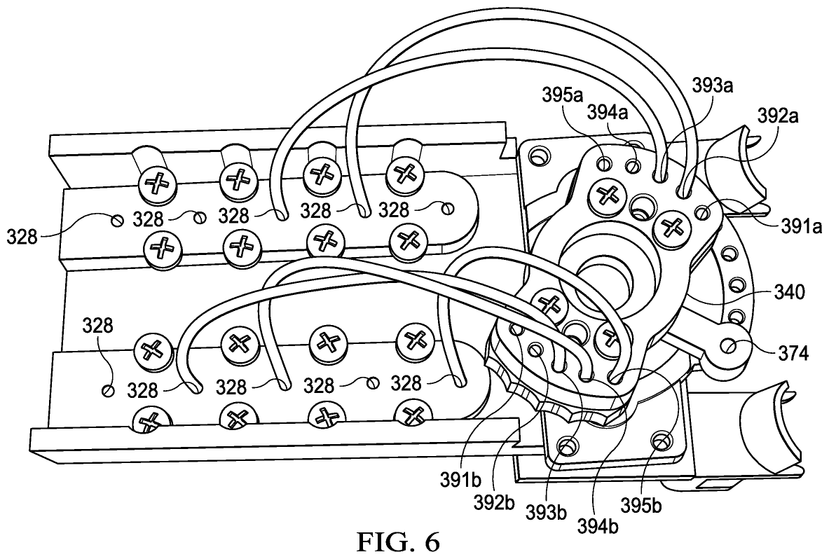

As shown in FIGS. 3-6, the representative system includes (in addition to the specimen processing cartridge 100) a nucleic acid processing device 300. The nucleic acid processing device 300 may be a 3-D printed structure formed by a Form 2 SLA printer and clear photopolymer resin material. Other fabrication techniques may also be suitable (e.g., machining, injection molding, and combinations and variations thereof).

An embodiment of an internal chassis 302 of the nucleic acid processing device 300 is described with regard to FIG. 3. The overall dimensions of the processing device 300 may be on the order of 12.5 cm×6 cm×3.5 cm (l×w×h) in a representative embodiment, though other sizes are possible. The nucleic acid processing device 300 includes an the internal chassis 302, which in turn includes a first filter housing 352 and a second filter housing 354. The filter housings may be cylindrical housings, and may be positioned on the internal chassis 302 proximate to an outlet of a valve or fluid flow mechanism (shown here as pivotable valve 340). The first filter housing 352 may be a DNA filter housing and the second filter housing 354 may be a RNA filter housing). In an illustrative embodiment, the filter housings are cylindrical chambers that are configured to house filters. The filters may be formed from silica binding membranes. In turn, the silica binding membranes are configured or selected to adsorb DNA and RNA, respectively, under various buffer and chaotropic salt conditions.

To form the filters, stacked silica discs (DNA silica discs and RNA silica discs) are loaded into each of the filter housings, along with polyethylene support discs. In an illustrative embodiment, the DNA silica discs and RNA silica discs are 9 mm in diameter and the DNA support disc and RNA support disc are approximately 1.5 mm thick. A DNA sample inlet port 314 and RNA sample inlet port 316 is positioned on each side of an internal chassis 302 the processing device 300 and is fitted with an inlet filter (e.g., a commercial 1.2 μm syringe filter that is 13 mm in diameter). Each inlet port may be used to accept a specimen recovered from, for example, the specimen processing cartridge 100 described above.

In an embodiment, the internal chassis 302 also includes one or more rows of buffer chambers. In the illustrative embodiment of FIG. 3, the rows of buffer chambers 330-339 are divided into a first row 320 of buffer chambers 330-334 and a second row 322 of buffer chambers 335-339. The buffer chambers 330-339 may be volumetric cylinders that contain up to 1 mL of wash or elution buffer. In some embodiments, the buffer chambers 330-339 may have a slightly conical shape and a fluidic outlet at their base.

Each of the buffer chambers 330-339 may be fitted with a rubber gasket lids. The gasket lids may act as buffer chamber actuators when depressed, and may be operable to motivate a stored fluid from the buffer chamber through the fluid outlet. Tubing segments or other suitable conduit may be used to form a liquid flow path from the fluidic outlets of the buffer chambers to an inlet of the pivotable valve 340 and to one of the filter housings. The tubing segments may be Tygon® tubing (non-DEHP, FDA approved 1/32″ ID and 3/32″ OD). In some embodiments, the buffer chambers 330-339 may be populated with fluids that are to be sequentially delivered to the first filter housing 352 and second filter housing 354.

In an illustrative embodiment, a first buffer chamber 330 of the first row 320 and first buffer chamber 335 of the second row may be arranged to be actuated at a first time, a second chamber 331 of the first row 320 and second buffer chamber 336 of the second row may be arranged to be actuated at a second time, a third buffer chamber 332 of the first row 320 and third buffer chamber 337 of the second row may be arranged to be actuated at a third time, and so on. The buffer chambers 330-339 may also be populated or left empty depending on whether it is desirable to deliver a fluid from the respective chamber to the respective filter housing. For example, in the configuration shown in FIG. 3, the first chamber 330 of the first row 320 is left empty whereas the first buffer chamber 335 of the second row 322 is populated so that with the first buffer chambers 330, 335 are actuated, fluid will be dispensed from the first buffer chamber 335 of the second row 322 to the second filter housing 354 but no fluid will be dispensed to the first filter housing 352.

The foregoing arrangement allows for a timed and sequenced delivery of fluids (generally liquids) from the respective buffer chambers 330-339 to the first filter housing 352 and second filter housing 354. In operation, buffer liquids may thereby be sequentially passed though the DNA silica discs and RNA silica discs of the filter housings by manually applying pressure to the gasket lids 356 using a push-button actuator 362.

The buffer chambers 330-339 and tubing may form a fluidic path from each of the buffer chambers 330-339 to the DNA/RNA binding membranes of the respective filter housings 352, 354. Fluid flow may be controlled by the pivotable valve 340. The pivotable valve 340 may be manually adjusted by the user or automatically indexed through positions that correspond to the desired flow paths during operation of the processing device 300. Between the first row 320 and second row 322 of buffer chambers 330-339 is a relatively large (e.g., 5 mL volume) central waste reservoir that receives buffer liquids that have been passed through one of the filter housings.

In an embodiment, a final DNA buffer chamber (e.g., 332) and a final RNA buffer chamber (e.g., 338) contains an elution buffer that is operable to pass through the DNA silica discs RNA silica discs, respectively, and release any bound nucleic acids to a collection chamber instead of to the waste reservoir. The collection chambers may be a DNA microfuge tube positioned in a first microfuge tube holder 370 and a RNA microfuge tube positioned in a second microfuge tube holder 372.

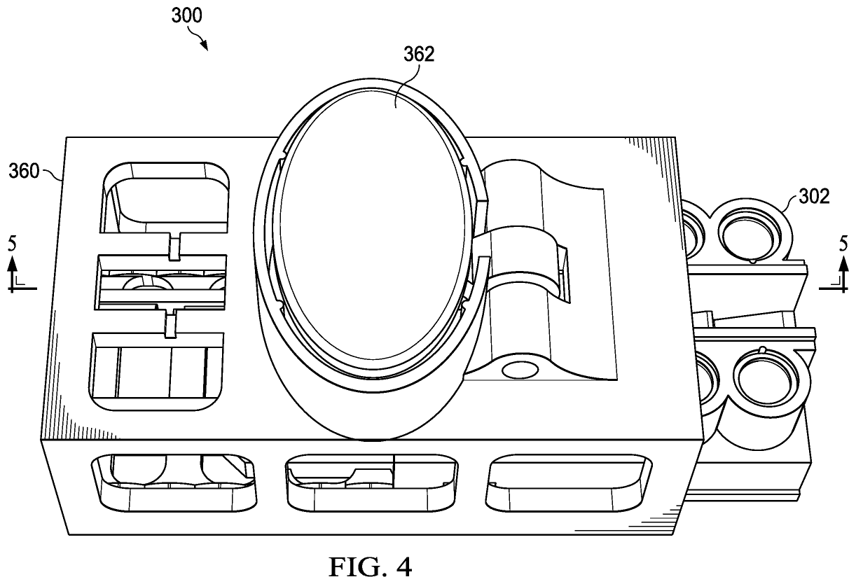

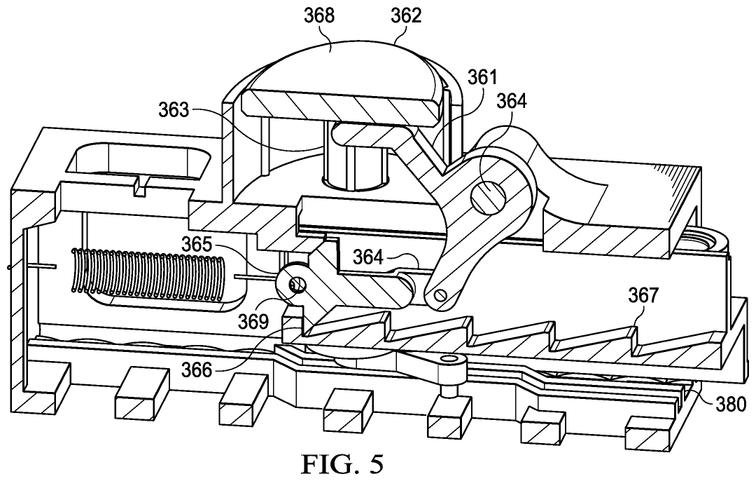

The processing device 300 may also include an external chassis 360, as shown in FIGS. 4 and 5. The external chassis 360 may be formed by molding, machining or using 3-D printing to receive and slidingly engage the internal chassis 302. The external chassis 360 may also act as a chassis for the other components of the nucleic acid processing device 300.

In some embodiments, the nucleic acid processing device 300 may be formed to minimize user involvement by allowing for a single, repeatable actuation step (i.e. one-button press) to sequentially dispense fluid from the respective buffer chambers 330-339. To facilitate operation using a single, repeatable actuation step, an advancing mechanism may be coupled to the processing structure's internal chassis 302 and external chassis 360. The advancing mechanism may enable one-way movement, or ratcheting, of the internal chassis 302 relative to the external chassis 360. In the embodiment shown in FIG. 4A, the advancing mechanism is a spring-loaded ratcheting mechanism. The spring-loaded ratcheting mechanism includes a first linkage member 361 that engages a first pivot 364 of the external chassis, and is operable to rotate about the first pivot 364 when an actuation surface 369 of the first linkage member 361 is depressed by a push-button actuator 362.

The push-button actuator 362 includes one or more pistons 363 that are sized and configured to engage and dispense fluid from a buffer chamber when the buffer chamber is aligned with the piston 363. The advancing mechanism also includes a second linkage member 358 that couples the first linkage member 361 to a third linkage member 365. The third linkage member 365, in turn is coupled to a spring that is fixed to the external chassis 360 at one end and an engagement feature of the third linkage member 365 (e.g., aperture 368). The third linkage member 365 also includes an engagement face 366 that is sized and configured to engage teeth 367 included on the internal chassis 302. Each tooth 367 of the internal chassis 302 corresponds to an index position wherein a buffer chamber (330-339) of the internal chassis 302 is aligned with the piston 363 of the push-button actuator 362.

In accordance with the foregoing, the processing device is arranged such that depressing the push-button actuator 362 will cause the piston(s) 363 to engage the buffer chambers below the push-button actuator 362 and dispense any fluid disposed therein through the respective filter housings. To facilitate the dispensing of fluid, each buffer chamber that is pre-filled with a fluid. Each buffer chamber may also include a buffer actuator 374 on the side of the buffer chamber that faces the piston 363. The buffer actuator 374 may be a grommet, lid and gasket (gasket lid), or similar component that slides within the buffer chamber to apply a compressive force to the contents of the buffer chamber. The opposing side of the buffer chamber may include a valve, such as a check valve, or frangible seal that acts as a fluidic outlet to allow fluid to be dispensed from the chamber when the buffer actuator 374 is depressed.

In addition to actuating the buffer actuator 374, the push-button actuator 362 also simultaneously actuates the advancing mechanism by engaging the first linkage member 361. In turn, the first linkage member 361 pulls the third linkage member 365 back to displace the engagement face 366 and deform the spring to engage the next sequential tooth 367. The foregoing action generates a spring force against the tooth to urge the internal chassis to the next index position. The internal chassis 302 is held static relative to the external chassis 360 while the push-button actuator 362 is depressed. When the push-button actuator 362 (which may be spring-loaded to return to its unactuated state) is released, the piston(s) 363 raise out of the underlying buffer chambers, thereby ceasing to oppose the spring force being applied to the internal chassis 302 through the engagement face 366 and allowing the internal chassis to slide to the next index position. Here, it is noted that certain mechanisms may also be included at the interface between the internal chassis 302 and internal chassis 360 to mark the index position. For example, complimentary indentations and protrusions or springed ball stops may be used to arrest motion of the internal chassis 302 when it reaches an index position.

Other advancing mechanisms may also be possible. For example, the push-button actuator 362 may be replaced with a power screw and dial, such that turning of the dial results in a comparable motion of pistons and movement of the internal chassis through a series of indexed detents or teeth to cause a similar process of ratcheting the internal chassis through a series of index positions.

As noted previously, the processing device 300 further includes the pivotable valve 340. The pivotable valve may be operated manually through a series of indexed positions, wherein each index position corresponds to buffer chamber position and a corresponding fluid path (created by the above-referenced tubing) from the buffer chamber(s) that are positioned below the piston(s) 363 to the filter housings. To that end, the pivotable valve 340 may include a plurality of valve inlets (391a-395a and 391b-395b) that sequentially align with the respective filter housing positioned below the pivotable valve 340. For example: (1) in a first index position in which first buffer chambers 330, 335 are aligned with the pistons 363, the pivotable valve 340 is rotated such that the first valve inlets 391a and 391b will form a fluid coupling from the tubing segments coupled to the exit valves of the first buffer chambers 330, 335 to the filter housings; (2) in a second index position in which second buffer chambers 331, 336 are aligned with the pistons 363, the pivotable valve 340 is rotated such that the second valve inlets 392a and 392b will form a fluid coupling from the tubing segments coupled to the exit valves of the second buffer chambers 331, 336 to the filter housings; (3) in a third index position in which third buffer chambers 332, 337 are aligned with the pistons 363, the pivotable valve 340 is rotated such that the third valve inlets 393a and 393b will form a fluid coupling from the tubing segments coupled to the exit valves of the third buffer chambers 332, 337 to the filter housings; (4) in a fourth index position in which fourth buffer chambers 333, 338 are aligned with the pistons 363, the pivotable valve 340 is rotated such that the fourth valve inlets 394a and 394b will form a fluid coupling from the tubing segments coupled to the exit valves of the fourth buffer chambers 333, 338 to the filter housings; and (4) in a fifth index position in which fifth buffer chambers 334, 339 are aligned with the pistons 363, the pivotable valve 340 is rotated such that the fifth valve inlets 395a and 395b will form a fluid coupling from the tubing segments coupled to the exit valves of the fifth buffer chambers 334, 339 to the filter housings.

The processing device 300 may be configured such that depressing of the push-button actuator 362 will result in moving the pivotable valve 340 through a sequence of positions that corresponds to sequential actuation of the buffer chambers. In such an embodiment, a full sequence of nucleic acid extraction steps may be achieved by pressing one button multiple (e.g., four) times after the introduction of the specimen.

In other embodiments, the pivotable valve 340 may be replaced with a fluidic network manifold located beneath the buffer chambers 330-339, which may streamline the extraction workflow while maintaining complete independence from external power or instrumentation.

In some embodiments, as shown in FIG. 1 the nucleic acid processing device may be formed integrally to the specimen processing device 100 to form an integrated system that is operable to process a nucleic acid specimen. Such a system would include a roiling chamber for receiving a sample collection device, an elution chamber, and a first actuator 102 for collecting the specimen from a swab or similar sample collection device. The specimen processing device would form and external chassis to house an internal chassis (similar to the embodiments described above) and include a second actuator 104 to actuate an internal nucleic acid processing device. The specimen processing device 100 may also include a DNA outlet port 106 for delivering a DNA sample to a DNA-purposed microfuge container and a RNA outlet port 108 for delivering a RNA sample to a RNA-purposed microfuge container.

In some embodiments, certain of the buffer chambers may serve as blanks (e.g., buffer chambers 330, 333, and 334), and therefore the first chamber 330 and second buffer chamber 331 may not be adjacent or sequentially arranged along the first row 320 of buffer chambers. The internal chassis 302 may also include a waste chamber that is coupled to a fluid outlet of the first filter housing 352 (which may be a DNA receiving chamber) and a fluid outlet of the second filter housing 354 (which may be an RNA receiving chamber) so that excess liquid from each buffer chamber may be drained from the respective filter housings to allow for preservation or further processing.

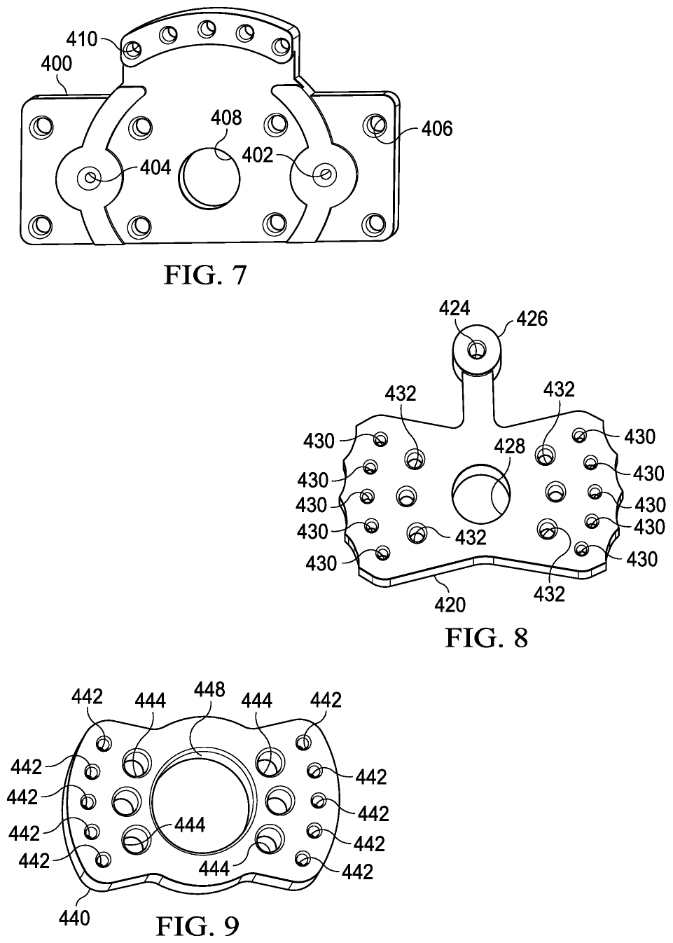

In some embodiments, the external chassis 360 may include a control track 380 and the valve 340 (See FIGS. 7-9) that includes a control pin inserted into a pinhole 374 of the valve 340, such that moving the internal chassis 302 relative to the external chassis 360 to a first index position causes the control pin to follow the control track 380 to orient the valve 340 in a first valve position in which the valve 340 fluidly couples the DNA receiving chamber to the first buffer chamber, and such that a first actuation of the actuator causes a piston to propel a first liquid from the first chamber 330 to the DNA receiving chamber.

The internal chassis 302 may be further operable to move relative to the external chassis 360 to a second index position in response to a second actuation of the actuator, which in turn causes the control pin to follow the control track 380 to orient the valve 340 in a second valve position in which the valve 340 fluidly couples the DNA receiving chamber (first filter housing 352) to the second buffer chamber 331, and wherein the second actuation of the actuator causes the piston to propel the second liquid from the second buffer chamber 331 to the DNA receiving chamber.

Components of a representative pivotable valve are shown in FIGS. 7-9, though sealing hardware and mounting hardware are not discussed here for brevity. FIG. 7 shows a valve base 400 that may be used to fix the valve to the internal chassis of the processing device. The base 400 includes one or more valve outlets (first valve outlet 402 and second valve outlet 404) which are operable to deliver liquid from the valve to the respective filter housings. In the embodiment shown, the valve base 400 also includes one or more mounting apertures 406 that may be used to mount the valve base 400 to the internal chassis, and a pivot aperture 408 that is operable to receive a rotational device, such as a bushing, bearing, pin, or similar device that facilitates rotation of the pivotable valve. The base also includes a plurality of index apertures 410 that are positioned to align with a guide-pin aperture of the valve when the valve is in a position that corresponds to an index position of the processing device.

FIG. 8 shows a tracking member 420 of the valve. The tracking member includes an intermediate pivot aperture 428 that is sized and configured to receive the rotational device referenced above. The tracking member 428 also includes a plurality of valve apertures 430 that may sequentially align with the valve outlet 402 of the valve base 400 as the internal chassis moves through the index positions discussed above. To operate the valve, the tracking member 420 includes a control arm 426 that can be manually operated by a user or fitted with a guide-pin at the pin aperture 424. In the latter instance, the guide-pin would engage the control track of the external chassis to control the valve automatically as the internal chassis moves through the external chassis. The tracking member also includes a plurality of mounting apertures 432 that are positioned to align with corresponding apertures of a tubing interface member.

FIG. 9 shows a representative interface member 440. In some embodiments, the interface member 440 and tracking member 420 may be formed as a combined, integral part. In the embodiment shown, the interface member includes an interface pivot aperture 448 that is sized and configured to receive the rotational device referenced above. The interface member 440 also includes a plurality of mounting apertures 444 that are positioned to align with the mounting apertures 432 of the tracking member 420. The interface member 440 also includes a plurality of valve inlet apertures 442 that are configured to engage fluid distribution conduits, such as the tubing segments described above, to receive fluid from the various buffer chambers when the processing device is operated.

With regard to operational aspects of a representative processing device, the various buffer chambers 330-339 may be preloaded with lysis buffers and reaction buffers to facilitate device operation. Additional materials may be provided in a kit, such as a suitable swab for sample extraction for the sample to be collected. The exemplary kit may also include two DNase/RNase-free microfuge tubes for collection of the eluted nucleic acids that is output from the nucleic acid processing device.

In subsequent processing, DNA/RNA extracts could be analyzed by A260/280 nm spectroscopy readings, such as a Take3 micro-volume plate on a Biotek Synergy™ H4 Hybrid plate reader.

In an illustrative embodiment, the disclosed nucleic acid processing device 300 may be deployed in a clinical setting in which a swab acquired sample can be obtained. Such settings may include (without limitation) an urgent care clinic, a doctor's office, or a field clinic in a remote location. A person with some medical training (i.e. Medical Assistant or Nurse) may be required to acquire the specimen, and the representative nucleic acid processing device may be operated by an individual having no medical training by virtue of the device's simplicity. It may be that an operator with a middle school or high school education could be trained to perform the extraction processing steps in less than 1 hour with 2 or more practice runs.

In the illustrative embodiments, no mixing or pipetting of sample or reagents is needed. Electric power may not be required to operate the device and all reagents may be pre-loaded/pre-metered, so that processing may be completed with minimal laboratory infrastructure. In fact, it may be that only a substantially flat solid surface or work area, such as a table top or bench, is needed to complete the extraction process.

Another advantage of the disclosed nucleic acid processing device is that the device provides a leak-proof biological seal for the sample with no potential for aerosol generation. This allows the sample to be safely processed without extensive personal protective equipment other than gloves without high risk of exposure to the sample.

Operational aspects of the nucleic acid processing system include the use of two separate devices for sample lysis and purification, though the devices may be integrated into a single device as described with regard to FIG. 1. Further, the system may be deployed in devices that are disposable or that are intended for re-use. In addition, as noted previously, the system may be implemented with a nucleic acid processing device that is manually operated with a push-button actuated chassis, fluid manifold and auto-advancement features described above, or in a more fully automated device that does not require repeated pressing of a button actuator. In addition, the specimen collection could be accomplished with clinical samples collected directly onto swabs, aliquoted into cryovials, or collected using another suitable process. In the case of obtaining a specimen from cryovials, pipetting steps may be necessary to get the appropriate volume of sample onto a swab. In test cases, hands-on processing time for the two devices was roughly 15 minutes, with a total turnaround time of 35-45 minutes per sample. All reaction buffers were prepared in advance and pre-loaded into the devices before beginning the extraction process as described below.

A representative process for cell lysis and recovery using the specimen processing cartridge is described below. To aid in cell lysis, 20 μL of Proteinase K was added to 80 μL of a thawed sample and allowed to incubate for 10-20 min. at room temperature. The sample was collected onto the swab by swirling a clean, sterile swab in the microfuge tube until all areas of the swab were visibly saturated (˜10-15 sec). The swab was then placed into the roiling chamber of the cartridge, the lid was closed, and the elution button pressed (<30 sec). The lysed sample (˜500 μL) was collected into a clean DNase/RNase-free microcentrifuge tube and then processed immediately using a nucleic acid processing device.

To purify the DNA and RNA using the nucleic acid processing device, the lysed sample taken directly from the roiling chamber was loaded into a 1 cc syringe and connected to the 1.2 μm syringe filter located at the inlet port of filter housing of the nucleic acid processing device. For stool samples, an additional 5 μm in-line filter was used to remove larger debris. The sample was passed through the first silica membrane, where DNA was adsorbed in the presence of chaotropic salts, using positive pressure from the syringe. Flow-through was directed into a volumetric chamber on the opposite side of the device containing 500 μL of 70% ethanol. The resulting sample plus ethanol mixture was then passed through a second silica membrane, which adsorbed RNA before going to waste, as the user manually pressed the gasket lid covering that chamber. The rotating valve was then twisted into the wash/waste position allowing for sequential delivery of two wash buffers to each of the DNA/RNA-bound membranes. Flow of the wash buffers was again initiated by depressing the buffer actuator on the appropriate buffer chamber. After washing, the membranes were allowed to air dry for 10 minutes. After drying, the valve was turned to the elution/recovery position and nuclease-free water was used to elute DNA from the first membrane followed by RNA from the second. Solubilized nucleic acids in water were collected into individual 1.5 mL microcentrifuge tubes.

Following extraction, each DNA/RNA eluted fraction was QC checked using an A260/280 spectrophotometric measurement on a Take3 micro-volume plate read on a Biotek Synergy™ H4 Hybrid plate reader. DNA/RNA fractions (at least 50 μL each) were pooled together and frozen at −80° C. for storage and shipment.

In order to maintain the overall device simplicity, lack of power requirement, and realistically keep the cost of goods low, the illustrative system does not have on-board data reporting or quality control evaluation methods. However, DNA/RNA extracts can easily be analyzed by A260/280 nm spectroscopy following the final elution step, prior to PCR (polymerase chain reaction) or downstream detection, as described above.

In accordance with the operational aspects of the foregoing system, an illustrative method of preparing a nucleic acid specimen may include depositing a lysed DNA sample to a DNA receiving chamber of a nucleic acid processing device. The nucleic acid processing device may include an actuator, and a first row of buffer chambers. The method may include activating an actuator of the of the nucleic acid processing device at a first time, wherein actuation of the actuator at the first time causes a first liquid to be propelled from a first chamber of the first row of buffer chambers to the DNA receiving chamber. The method may also include activating the actuator of the of the nucleic acid processing device at a second time, wherein actuation of the actuator at the second time causes a second liquid to be propelled from a second chamber of the first row of buffer chambers to the DNA receiving chamber. The first liquid may be 70-80% ETOH, and the second liquid may be a RNA stabilization solution.

In some embodiments, the nucleic acid processing device comprises an external chassis, an internal chassis, an actuator coupled to the external chassis and a piston for engaging a buffer chamber of the first row of buffer chambers, a pivotable valve coupled to the internal chassis, and a ratcheting mechanism. The internal chassis slidably engages the external chassis and the ratcheting mechanism is operable to move the internal chassis by an incremental distance relative to the external chassis each time the ratcheting mechanism is actuated. The pivotable valve is operable to fluidly couple the DNA receiving chamber the first buffer chamber at the first time, and to the second buffer chamber at the second time.

The external chassis may include a control track and the pivotable valve may include a corresponding control pin that engages the control track. In such embodiments, a first actuation of the actuator causes the internal chassis to move relative to the external chassis to a first index position, which in turn causes the control pin to follow the control track to orient the pivotable valve in a first position in which the pivotable valve fluidly couples the DNA receiving chamber to the first buffer chamber. The first actuation of the actuator may also cause the piston to propel the first liquid from the first buffer chamber to the DNA receiving chamber.

In some instances, a second actuation of the actuator causes the internal chassis to move relative to the external chassis to a second index position, which in turn causes the control pin to follow the control track to orient the pivotable valve in a second position in which the pivotable valve fluidly couples the DNA receiving chamber to the second buffer chamber. The second actuation of the actuator may similarly cause the piston to propel the second liquid from the second buffer chamber to the DNA receiving chamber.

In embodiments in which RNA samples are processed in parallel with the DNA sample, the method may further include depositing a lysed RNA sample to a RNA receiving chamber of a nucleic acid processing device. Here, the nucleic acid processing device further includes a second row of buffer chambers, and the device is configured such that actuation of the actuator at a third time (which may be prior to the second time referenced above) causes a third liquid to be propelled from a third chamber of the second row of buffer chambers to the RNA receiving chamber. Actuating the actuator of the of the nucleic acid processing device at the second time referenced above may cause a fourth liquid to be propelled from a fourth chamber of the second row of buffer chambers to the RNA receiving chamber, and activating the actuator of the of the nucleic acid processing device at a fourth time, which may be after the third time, causes a fifth liquid to be propelled from a fifth chamber of the second row of buffer chambers to the RNA receiving chamber. Here, the third liquid may be an organic solvent, the fourth liquid may be 70-80% ETOH, and the fifth liquid may be a stabilization solution.

In some embodiments, the pivotable valve is operable to fluidly couple the RNA receiving chamber to the third buffer chamber at the third time, to the fourth buffer chamber at the second time, and to the fifth buffer chamber at the fourth time. Further, to facilitate parallel processing, the actuator may be further coupled to a second piston for engaging a buffer chamber of the second row of buffer chambers. In such embodiments, causing the internal chassis to move relative to the external chassis to the third index position (which may be, sequentially, prior to the first index position described above) may in turn cause the control pin to track the control track and orient the pivotable in a configuration that fluidly couples the RNA receiving chamber to the third buffer chamber. Here, the third actuation of the actuator may cause the second piston to propel the third liquid from the third buffer chamber to the RNA receiving chamber.

In some instances, the second actuation of the actuator causes the internal chassis to move relative to the external chassis to the second index position, which in turn causes the control pin to follow the control track to orient the pivotable valve in the position in which the pivotable valve fluidly couples the RNA receiving chamber to the fourth buffer chamber, and the second piston to propel the fourth liquid from the fourth buffer chamber to the RNA receiving chamber.

Similarly, a fourth actuation of the actuator causes the internal chassis to move relative to the external chassis to a fourth index position, which in turn causes the control pin to follow the control track to orient the pivotable valve in a fourth position in which the pivotable valve fluidly couples the RNA receiving chamber to the fifth buffer chamber, and wherein the fourth actuation of the actuator results in the second piston propelling the fifth liquid from the fifth buffer chamber to the RNA receiving chamber.

It is noted that unless an embodiment is expressly stated as being incompatible with other embodiments, the concepts and features described with respect to each embodiment may be applicable to and applied in connection with concepts and features described in the other embodiments without departing from the scope of this disclosure. To that end, the above-disclosed embodiments have been presented for purposes of illustration and to enable one of ordinary skill in the art to practice the disclosure, but the disclosure is not intended to be exhaustive or limited to the forms disclosed. Many insubstantial modifications and variations will be apparent to those of ordinary skill in the art without departing from the scope and spirit of the disclosure. The scope of the claims is intended to broadly cover the disclosed embodiments and any such modification.

As used herein, the singular forms “a”, “an” and “the” are intended to include the plural forms as well, unless the context clearly indicates otherwise. It will be further understood that the terms “comprise” and/or “comprising,” when used in this specification and/or the claims, specify the presence of stated features, steps, operations, elements, and/or components, but do not preclude the presence or addition of one or more other features, steps, operations, elements, components, and/or groups thereof. In addition, the steps and components described in the above embodiments and figures are merely illustrative and do not imply that any particular step or component is a requirement of a claimed embodiment.

1. A method of preparing a nucleic acid specimen, the method comprising:
depositing a lysed DNA sample to a DNA receiving chamber of a nucleic acid processing device, the nucleic acid processing device comprising an actuator, and a first row of buffer chambers;
activating the actuator of the of the nucleic acid processing device at a first time, wherein actuation of the actuator at the first time causes a first liquid to be propelled from a first chamber of the first row of buffer chambers to the DNA receiving chamber; and
activating the actuator of the of the nucleic acid processing device at a second time, wherein actuation of the actuator at the second time causes a second liquid to be propelled from a second buffer chamber of the first row of buffer chambers to the DNA receiving chamber.

2. The method of claim 1, wherein the first liquid comprises 70-80% ETOH.

3. The method of claim 1, wherein the second liquid comprises a DNA stabilization solution.

4. The method of claim 1, wherein the nucleic acid processing device comprises an external chassis, an internal chassis, an actuator coupled to the external chassis and a piston for engaging a buffer chamber of the first row of buffer chambers, a valve coupled to the internal chassis, and a ratcheting mechanism, wherein the internal chassis slidably engages the external chassis, wherein the ratcheting mechanism is operable to move the internal chassis by an incremental distance relative to the external chassis each time the ratcheting mechanism is actuated, and wherein the valve is operable to fluidly couple the DNA receiving chamber to one the first buffer chamber at the first time, and to the second buffer chamber at the second time.

5. The method of claim 4, wherein the external chassis comprises a control track and wherein the valve comprises a control pin that engage the control track, and wherein a first actuation of the actuator causes the internal chassis to move relative to the external chassis to a first index position, which in turn causes the control pin to follow the control track to orient the valve in a first position in which the valve fluidly couples the DNA receiving chamber to the first buffer chamber, and wherein the first actuation of the actuator causes the piston to propel the first liquid from the first buffer chamber to the DNA receiving chamber.

6. The method of claim 5, wherein a second actuation of the actuator causes the internal chassis to move relative to the external chassis to a second index position, which in turn causes the control pin to follow the control track to orient the valve in a second position in which the valve fluidly couples the DNA receiving chamber to the second buffer chamber, and wherein the second actuation of the actuator causes the piston to propel the second liquid from the second buffer chamber to the DNA receiving chamber.

7. The method of claim 6, further comprising:
depositing a lysed RNA sample to a RNA receiving chamber of a nucleic acid processing device, the nucleic acid further comprising a second row of buffer chambers, wherein actuation of the actuator at the second time causes a third liquid to be propelled from a third buffer chamber of the second row of buffer chambers to the RNA receiving chamber;
activating the actuator of the of the nucleic acid processing device at a third time, wherein actuation of the actuator at the third time causes a fourth liquid to be propelled from a fourth buffer chamber of the second row of buffer chambers to the RNA receiving chamber; and
activating the actuator of the of the nucleic acid processing device at a fourth time, wherein actuation of the actuator at the fourth time causes a fifth liquid to be propelled from a fifth buffer chamber of the second row of buffer chambers to the RNA receiving chamber.

8. The method of claim 7, wherein the third liquid comprises an organic solvent.

9. The method of claim 7, wherein the fourth liquid comprises 70-80% ETOH.

10. The method of claim 7, wherein the fifth liquid comprises a stabilization solution.

11. The method of claim 7, wherein the nucleic acid processing device comprises a valve that is operable to fluidly couple the RNA receiving chamber to one the third buffer chamber at the second time, to the fourth buffer chamber at the third time, and to the fifth buffer chamber at the fourth time.

12. The method of claim 11, wherein the actuator is further coupled to a second piston for engaging a buffer chamber of the second row of buffer chambers, wherein causing the internal chassis to move relative to the external chassis to the second index position, which in turn causes the control pin to fluidly couple the RNA receiving chamber to the third buffer chamber, and wherein the second actuation of the actuator causes the second piston to propel the third liquid from the third buffer chamber to the RNA receiving chamber.

13. The method of claim 12, wherein a third actuation of the actuator causes the internal chassis to move relative to the external chassis to a third index position, which in turn causes the control pin to follow the control track to orient the valve in a third position in which the valve fluidly couples the RNA receiving chamber to the fourth buffer chamber, and wherein the third actuation of the actuator causes the second piston to propel the fourth liquid from the fourth buffer chamber to the RNA receiving chamber.

14. The method of claim 13, wherein a fourth actuation of the actuator causes the internal chassis to move relative to the external chassis to a fourth index position, which in turn causes the control pin to follow the control track to orient the valve in a fourth position in which the valve fluidly couples the RNA receiving chamber to the fifth buffer chamber, and wherein the fourth actuation of the actuator results in a liquid, and wherein the second actuation of the actuator causes the second piston to propel the fifth liquid from the fifth buffer chamber to the RNA receiving chamber.

15. A sample processing device comprising:
an internal chassis comprising a first receiving chamber and a first row of buffer chambers;
an external chassis comprising an actuator, the actuator being operable to actuate a piston that is operable to engage each buffer chamber of the first row of buffer chambers;
a valve coupled to the internal chassis and being operable to fluidly couple the first receiving chamber to a first buffer chamber of the first row of buffer chambers when the valve is in a first orientation, and to a second buffer chamber when the valve is in a second orientation; and
a lever that is operable to move the internal chassis relative to the external chassis though a plurality of index positions in response to the actuator being activated,
wherein the actuator is operable to cause a first liquid to be propelled from the first buffer chamber to the first receiving chamber when actuated at a first time.

16. The sample processing device of claim 15, wherein the external chassis comprises a control track and wherein the valve comprises a control pin that is operable to engage the control track and follow the control track to orient the valve in a first valve position in which the valve fluidly couples the first receiving chamber to the first buffer chamber when the internal chassis is in a first index position relative to the external chassis.

17. The sample processing device of claim 16, wherein the internal chassis is operable to move relative to the external chassis to a second index position in response to actuation of the actuator, and wherein the control pin is operable to follow the control track to orient the valve in a second valve position in which the valve fluidly couples the first receiving chamber to the second buffer chamber when the internal chassis is in a second index position relative to the external chassis, and wherein the actuator is operable cause the piston to propel a second liquid from the second buffer chamber to the first receiving chamber in response to a second actuation of the actuator.

18. The sample processing device of claim 16, wherein the first receiving chamber comprises a DNA filter housing.

19. The sample processing device of claim 16,
wherein the internal chassis further comprises a second receiving chamber and a second row of buffer chambers;
wherein the actuator is further operable to actuate a second piston that is operable to engage each buffer chamber of the second row of buffer chambers;
wherein the valve is further operable to fluidly couple the second receiving chamber to a first buffer chamber of the second row of buffer chambers when the internal chassis is in the first index position relative to the external chassis, and
wherein activating the actuator at the first time causes a third liquid to be propelled from the first buffer chamber of the second row of buffer chambers to the second receiving chamber.

20. The sample processing device of claim 19, wherein the second receiving chamber comprises a RNA filter housing.
